(19) United States
(12) Patent Application Publication
(10) Pub. No.: US 2024/0057900 A1
(43) Pub. Date: Feb. 22, 2024

(54) SYSTEMS, METHODS AND APPARATUS FOR GENERATING BLOOD GLUCOSE ESTIMATIONS USING REAL-TIME PHOTOPLETHYSMOGRAPHY DATA

(71) Applicant: VALENCELL, INC., Raleigh, NC (US)

(72) Inventors: Namita LOKARE, Raleigh, NC (US); Tushar Dilip TANK, Raleigh, NC (US); Steven Francis LEBOEUF, Raleigh, NC (US); Keith RICHESON, Raleigh, NC (US)

(21) Appl. No.: 18/256,024

(22) PCT Filed: Dec. 23, 2021
(86) PCT No.: PCT/US2021/065119

§ 371 Date: Jun. 5, 2023

Related U.S. Application Data
(60) Provisional application No. 63/132,233, filed on Dec. 30, 2020.

(51) Int. Cl. A61B 5/145; A61B 5/0205; A61B 5/00; G16H 50/70

(52) U.S. Cl. CPC A61B 5/14532; A61B 5/0205; A61B 5/7275; G16H 50/70; A61B 5/02416

(57) ABSTRACT

A method of generating a blood glucose estimation for a subject includes receiving real-time PPG data from a PPG sensor attached to the subject, and generating a blood glucose estimation for the subject via an adaptive predictive model using the real time PPG data. The method includes receiving a real-time measurement of blood glucose via a blood glucose monitoring device attached to the subject and, in response to receiving the real-time measurement of blood glucose, updating one or more parameters of the model in real-time to improve blood glucose estimation accuracy of the model. The method may also include detecting whether the generated blood glucose estimation is above or below a threshold and, in response to determining that the generated blood glucose estimation is above or below the threshold, receiving a real-time measurement of blood glucose and then updating the parameters of the model to improve blood glucose estimation accuracy of the model.

### RELATED APPLICATION

This application claims the benefit of and priority to U.S. Provisional Patent Application No. 63/132,233 filed Dec. 30, 2020, the disclosure of which is incorporated herein by reference as if set forth in its entirety.

### FIELD OF THE INVENTION

The present invention relates generally to wearable devices, and more particularly to wearable biometric sensor technology for physiological monitoring for medical, health, and fitness applications.

### BACKGROUND OF THE INVENTION

The “holy grail” of diabetes management would comprise a truly noninvasive, continuous blood glucose monitoring solution that would be completely painless and nearly invisible to the end user. Many attempts have been made to provide such a commercial solution, but none have succeeded.

Continuous blood glucose monitoring solutions are commercially available in the marketplace today, such as the Dexcom G6 CGM system (Dexcom, Inc., San Diego, California). These conventional monitoring systems take regular samples of body fluids (such as interstitial fluid), via microneedles or other minimally invasive modalities, and estimate blood glucose from sensor readings. However, by nature they are at best minimally invasive, and typically cause agitation to the underlying skin. Moreover, the concentration of glucose in interstitial fluid typically lags that of blood by several minutes, which may delay urgent feedback to the end user. Of equal importance, the form-factor of conventional glucose monitoring systems is typically that of a patch, which may feel awkward to many end users.

As reported by John Smith in “The Pursuit of Noninvasive Glucose: Hunting the Deceitful Turkey”, a myriad of researchers have labored over a noninvasive method of noninvasively measuring blood glucose levels accurately enough to dose insulin or glucose. However, the results have not been suitable for commercial use. A novel approach to this problem is needed.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

According to some embodiments of the present invention, a method of generating a blood glucose estimation for a subject includes the following steps performed by at least one processor: receiving real-time PPG data from a PPG sensor attached to the subject, and generating a blood glucose estimation for the subject via an adaptive predictive model using the real time PPG data. Exemplary adaptive predictive models include, but are not limited to, regression models, machine learning models, and classifier models. Generating the blood glucose estimation may include generating a current blood glucose estimation. Generating the blood glucose estimation may include generating a future blood glucose estimation. In some embodiments, a current blood glucose estimation and a past blood glucose estimation may be processed to predict a future blood glucose estimation.

The method may further include receiving a measurement of blood glucose from a blood glucose monitoring device and, in response to receiving the measurement of blood glucose, updating one or more parameters of the adaptive predictive model in real-time to improve blood glucose estimation accuracy of the adaptive predictive model. In some embodiments, the measurement of blood glucose is a real-time measurement.

The method may further include receiving subject activity information from a motion sensor attached to the subject, receiving a real-time measurement of blood glucose from a blood glucose monitoring device in response to the subject activity information and, in response to receiving the real-time measurement of blood glucose, updating one or more parameters of the adaptive predictive model in real-time to improve blood glucose estimation accuracy of the adaptive predictive model.

The method may further include detecting whether the generated blood glucose estimation is above or below a threshold. In response to determining that the generated blood glucose estimation is above or below the threshold, the method further includes receiving a measurement of blood glucose via a blood glucose monitoring device, and updating one or more parameters of the adaptive predictive model in real-time to improve blood glucose estimation accuracy of the adaptive predictive model.

In some embodiments, the method further includes sending an alert to a remote device that the generated blood glucose estimation is above or below a threshold.

In some embodiments, the at least one processor is located within a wearable device worn by the subject. The wearable device may be configured to be worn at an ear of the subject, on a limb of the subject, as a patch attached to the subject, or on a digit of the subject.

In some embodiments, the wearable device includes the PPG sensor.

In some embodiments, the at least one processor is located within a wearable device worn by the subject, and the wearable device includes the PPG sensor and the blood glucose monitoring device.

In some embodiments, the PPG sensor is an imaging sensor.

According to other embodiments of the present invention, a wearable device includes a PPG sensor and at least one processor configured to generate a blood glucose estimation for a subject wearing the wearable device via an adaptive predictive model using real-time PPG data from the PPG sensor. The wearable device may be configured to be worn at an ear of the subject, on a limb of the subject, as a patch attached to the subject, or on a digit of the subject. Exemplary adaptive predictive models may include, but are not limited to, regression models, machine learning models, and classifier models.

The at least one processor may be configured to receive a measurement of blood glucose from a blood glucose monitoring device and, in response to receiving the real-time measurement of blood glucose, update one or more parameters of the adaptive predictive model in real-time to improve blood glucose estimation accuracy of the adaptive predictive model. The at least one processor may be configured to receive a measurement of blood glucose from a blood glucose monitoring device and, in response to determining that the generated blood glucose estimation is above or below a threshold, update one or more parameters of the adaptive predictive model in real-time to improve blood glucose estimation accuracy of the adaptive predictive model. The at least one processor may be configured to send an alert to a remote device that the generated blood glucose estimation is above or below a threshold.

In some embodiments, the PPG sensor is an imaging sensor.

According to other embodiments of the present invention, a method of improving blood glucose estimation accuracy of an adaptive predictive model (e.g., a regression model, a machine learning model, a classifier model, etc.) includes the following steps performed by at least one processor: a) receiving, within a receiving period, real-time PPG data from a PPG sensor attached to a subject and a blood glucose measurement from a blood glucose monitoring; b) generating features from the received PPG data; c) storing the features and the blood glucose measurement; and d) updating one or more parameters of the adaptive predictive model in real-time by processing the stored features in context with the stored blood glucose measurement, wherein the updated one or more parameters improves the blood glucose estimation accuracy of the adaptive predictive model. The features and the blood glucose measurement may be stored in a data buffer, such as a FIFO (first-in-first-out) buffer, although other types of data buffers may be utilized. Steps a)-d) may be repeated over one or more subsequent time periods to improve estimation accuracy of the model.

The method may include generating a blood glucose estimation for the subject via the adaptive predictive model, then determining whether the generated blood glucose estimation is above or below a threshold. Responsive to determining that the generated blood glucose estimation is above or below a threshold, another measurement of blood glucose via the blood glucose monitoring device is received, and the one or more parameters of the adaptive predictive model are updated in real-time.

In some embodiments, generating features from the received PPG data includes generating features at feature generation intervals within the receiving period via a sliding time window. In some embodiments, updating the one or more parameters of the adaptive predictive model further includes processing the stored blood glucose measurement and a previously stored blood glucose measurement, and generating an interpolation between the stored blood glucose measurement and the previously stored blood glucose measurement. Processing the stored blood glucose measurement and the previously stored blood glucose measurement may include processing a plurality of previously stored blood glucose measurements. Processing the stored blood glucose measurement and the previously stored blood glucose measurement may include generating an interpolation of expected blood glucose measurements.

In some embodiments, the PPG sensor is an imaging sensor.

In some embodiments, processing the stored features in context with the stored blood glucose measurement includes processing a function of at least one of the stored features.

In some embodiments, processing the stored features in context with the stored blood glucose measurement includes calculating statistical information for a temporal sequence of at least one of the stored features. In some embodiments, processing the stored features in context with the stored blood glucose measurement includes calculating statistical information for a plurality of temporal sequences of at least one of the stored features.

In some embodiments, processing the stored features in context with the stored blood glucose measurement includes calculating weighted statistical information for a plurality of temporal sequences of at least one of the stored features.

According to other embodiments of the present invention, a system for improving blood glucose estimation accuracy of an adaptive predictive model (e.g., a regression model, a machine learning model, a classifier model, etc.) includes at least one processor configured to: receive, within a receiving period, real-time PPG data from a PPG sensor attached to a subject and a blood glucose measurement from a blood glucose monitoring device; generate features from the received PPG data; store the features and the blood glucose measurement; and update one or more parameters of the adaptive predictive model in real-time by processing the stored features in context with the stored blood glucose measurement, wherein the updated at least one parameter improves blood glucose estimation accuracy of the adaptive predictive model.

In some embodiments, the at least one processor is further configured to: generate a blood glucose estimation via the adaptive predictive model; determine whether the generated blood glucose estimation is above or below a threshold; and in response to determining that the generated blood glucose estimation is above or below the threshold, update the one or more parameters of the adaptive predictive model in real-time. The at least one processor may be configured to send an alert to a remote device that the generated blood glucose estimation is above or below the threshold.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate various embodiments of the present invention. The drawings and description together serve to fully explain embodiments of the present invention.

FIG. 1 illustrates a computational system for generating biometric estimations, according to some embodiments of the present invention.

FIGS. 2-4 are flowcharts of methods of generating biometric estimations, according to some embodiments of the present invention.

FIG. 5 illustrates exemplary, non-limiting, wearable devices that may be utilized in accordance with embodiments of the present invention.

FIG. 6 illustrates a sliding window of time that may be utilized to receive PPG data and biometric data, according to some embodiments of the present invention.

FIG. 7 is a block diagram illustrating operations for updating one or more parameters of an adaptive predictive model, according to some embodiments of the present invention.

FIG. 8 illustrates an adaptive predictive model, according to some embodiments of the present invention.

FIG. 9 illustrates a computational system for generating biometric estimations, according to some embodiments of the present invention.

FIG. 10 is a flowchart of methods of generating biometric estimations, according to some embodiments of the present invention.

FIG. 11 is a block diagram illustrating operations for updating one or more parameters of an adaptive predictive model, according to some embodiments of the present invention.

FIG. 12 is a data plot collected from a subject wearing a blood pressure cuff and a PPG sensor, and illustrating the collection of real-time BP measurement data and real-time PPG-BP estimations, according to some embodiments of the present invention.

FIG. 13 is a graphic output of estimated blood pressure and actual blood pressure for a subject over a time period and illustrating improvement of blood pressure estimation over time via augmentation.

FIG. 14 illustrates tables comparing volume clamp BP estimations with PPG-BP estimates according to embodiments of the present invention, in terms of accuracy with respect to actual BP measurements.

FIG. 15 is a block diagram that illustrates details of an exemplary processor and memory that may be used in accordance with various embodiments of the present invention.

### DETAILED DESCRIPTION

The term “subject”, as used herein, typically refers to a human being in context of the invention description. However, in context of the invention, a subject may also be a living creature that is not a human being.

The term “biometric” generally refers to a metric for a subject generated by processing physiological (i.e., biological) information from the subject. Nonlimiting examples of biometrics may include: heart rate (HR), heart rate variability (HRV), RR-interval, respiration rate, weight, height, sex, physiological status, overall health status, disease conditions, injury status, blood pressure, arterial stiffness, cardiovascular fitness, VO₂max, gas exchange analysis metrics, blood analyte levels fluid metabolite levels, and the like.

The terms “biometric” and “physiological metric”, as used herein are interchangeable.

The term “real-time” is used herein to describe a process that requires a period of time that appears substantially real-time to a human individual. Thus, the term “real-time” is used interchangeably to mean “near real-time” or “quasi-real-time”. Namely, a “real-time” process may refer to an “instantaneous process” but may also refer to a process that generates an output within a short enough processing time to (in effect) be as useful as an instantaneous process (in context of a particular use case). For example, in practicality, a process that requires several seconds or minutes to generate a blood pressure metric for a subject may be considered to be a real time process, as used herein, even though blood pressure may be changing each second, as the use case may involve a sedentary state for the subject where subtle changes in blood pressure may be insignificant and averaged out.

The terms “respiration rate” and “breathing rate”, as used herein, are interchangeable.

The terms “heart rate” and “pulse rate”, as used herein, are interchangeable.

The term “system”, as used herein, refers to a collection of physical and/or computational materials that may be unified by a common function.

The terms “motion sensor”, as used herein, refers to a sensor configured to sense motion information (e.g., from a subject). Nonlimiting examples of motion sensors may comprise: single- or multi-axis inertial sensors (such as accelerometers, gyroscopes, MEMS motion sensors, and the like), optical scatter sensors, blocked channel sensors, and the like.

The term “photoplethysmography” (PPG), as used herein, refers to a method of generating physiological information from PPG waveforms collected via a PPG sensor.

The term “PPG waveform”, as used herein, refers to physiological waveform data resulting from a temporal modulation of photon flux through physiological material.

The term “PPG sensor”, as used herein, refers to a sensor configured to sense photons and generate PPG waveform data. A typical PPG sensor may comprise an optical sensor configured to sense photons in the optical spectrum (i.e., an electromagnetic wavelength range of ˜10 nm to 103 μm, or electromagnetic frequencies in the range from ˜300 GHz to 3000 THz). Nonlimiting examples of optical sensors may comprise inorganic and/or organic photodetectors (such as photoconductors, photodiodes, phototransistors, phototransducers, and the like), reverse-biased light-emitting diodes (LEDs) or other reverse-biased optical emitters, imaging sensors, photodetector arrays, and the like. Additionally, a typical PPG sensor may also comprise a photon (photonic) emitter to generate a photon flux through a physiological pathway. However, in some cases, ambient photons or photons from another source (that is not part of the PPG sensor) may be used to generate photons. Typical PPG sensors may comprise photon emitters that are optical emitters, such as inorganic and/or organic light-emitting diodes (LEDs), laser diodes (LDs), microplasma sources, or the like. PPG sensors may also comprise a motion sensor for the purposes of generating subject activity data and/or providing a noise reference for attenuating motion artifacts in PPG waveform data.

The terms “sensor”, “sensing element”, and “sensor module”, as used herein, are interchangeable and refer to a sensor element or group of sensor elements that may be utilized to sense information, such as information (e.g., physiological information, body motion, etc.) from the body of a subject and/or environmental information in a vicinity of the subject. A sensor/sensing element/sensor module may comprise one or more of the following: a detector element, an emitter element, a processing element, optics, or optomechanics, sensor mechanics, mechanical support, supporting circuitry, and the like. Both a single sensor element and a collection of sensor elements may be considered a sensor, a sensing element, or a sensor module. A sensor/sensing element/sensor module may be configured to both sense information and process that information into one or more metrics.

As used herein, the term “processor” broadly refers to a signal processing circuit or computing system, or a computational method, which may be localized and/or distributed. For example, a localized signal processing circuit may comprise one or more signal processing circuits or processing methods localized to a general location, such as to a wearable biometric monitoring device. Examples of such devices may comprise, but are not limited to, an earpiece, a headpiece, a finger clip, a toe clip, a limb band (such as an arm band or leg band), an ankle band, a wrist band, a digit (e.g., finger or toe) band, a nose band, a sensor patch, jewelry, a patch, apparel (clothing) or the like. Examples of a distributed processing circuit include “the cloud,” the internet, a remote database, a remote processor computer, a plurality of remote processing circuits or computers in communication with each other, etc., or processing methods distributed among one or more of these elements. The difference between distributed and localized processing circuits is that a distributed processing circuit may include delocalized elements, whereas a localized processing circuit may work independently of a distributed processing system. Microprocessors, microcontrollers, or digital signal processing circuits represent a few non-limiting examples of signal processing circuits that may be found in a localized and/or distributed system.

The terms “mobile application”, “mobile app” and “app”, as used herein, are interchangeable and refer to a software program that can run on a computing apparatus, such as a mobile phone, digital computer, smartphone, database, cloud server, processor, wearable device, or the like.

The term “health”, as used herein, is broadly construed to relate to the physiological status of an organism or of a physiological element or process of an organism. For example, cardiovascular health may refer to the overall condition of the cardiovascular system, and a cardiovascular health assessment may refer to an estimate of blood pressure, VO₂max, cardiac efficiency, heart rate recovery, arterial blockage, arrhythmia, atrial fibrillation, or the like. A “fitness” assessment is a subset of a health assessment, where the fitness assessment refers to how one's health affects one's performance at an activity. For example, a VO₂max test can be used to provide a health assessment of one's mortality or a fitness assessment of one's ability to utilize oxygen during an exercise.

The term “blood pressure”, as used herein, refers to a measurement or estimate of the pressure associated with blood flow of a person, such as a diastolic blood pressure, a systolic blood pressure, a mean arterial pressure, pulse pressure, or the like. The blood pressure may be with reference to any location on the body where blood vessels and blood flow exists (i.e., brachial, thoracic, subclavian, femoral, tibial, radial, carotid, and the like). The term “blood pressure” is abbreviated as “BP” throughout this document.

As used herein, any device or system is considered to be remote to another device or system as long as there is no physical connection between them. As a point of clarity, the term “remote” does not necessarily mean that a remote device is a wireless device or that it is a long distance away from a device in communication therewith. For example, in some cases, two devices may be considered remote devices with respect to each other even if there is a physical connection between them. In this case, the term “remote” is intended to reference a device or system that is distinct from another device or system or that is not substantially reliant on another device or system for core functionality. For example, a computer wired to a wearable device may be considered a remote device, as the two devices are distinct and/or not substantially reliant on each other for core functionality.

The terms “sampling frequency”, “signal analysis frequency”, and “signal sampling rate”, as used herein, are interchangeable and refer to the number of samples per second (or per other time unit) taken from a continuous sensor or sensing element (for example, the sampling rate of the thermopile output in a tympanic temperature sensor or the sampling rate of the PPG signal from a PPG sensor).

It should be noted that “algorithm” and “circuit” are referred to herein. An algorithm refers to a computational instruction set, such as an instruction set with sequential steps and logic, that may be in memory whereas a circuit refers to physical components and/or traces (or path) that may implement such logic operations in the digital, analog, and/or quantum domains. These circuits may typically comprise electric circuits but may alternatively comprise elements that are photonic, electromagnetic, magnetic, acoustic, quantum, or the like.

To address these limitations, methods and apparatus according to the present invention provide for continuously generating blood pressure estimates (and/or other biometric estimates including, but not limited to, EEG estimates, respiratory metric estimates, core body temperature estimates, blood glucose estimates, etc.) via a real-time adaptive predictive model. These methods and apparatus leverage continuous PPG measurements from a subject, combined with at least one BP (or other biometric) measurement from a subject, to update, in real-time, a predictive model for that subject that is more accurate in estimating BP (or other biometric) for that subject (than prior to the update). The methods of the present invention may be implemented in a computational system that is configured to receive the PPG and BP (or other biometric) data and process this data to improve estimation accuracy. Namely, the model may be configured to generate a BP estimate for a given set of PPG input features, such that the BP estimate is a function of the PPG features, and the parameters of the model may be updated over time as recurring BP measurements (e.g., from a cuff-based BP monitor) are processed to improve the error of the model. The PPG sensors may be wearable and thus integrated into an apparatus or material that is in proximity to the skin of the subject. Alternatively, the PPG sensors may be stand-off sensors, such as imaging sensors (e.g., cameras) remote scanning sensors (e.g., radar, doppler, etc.), or the like, as described later herein.

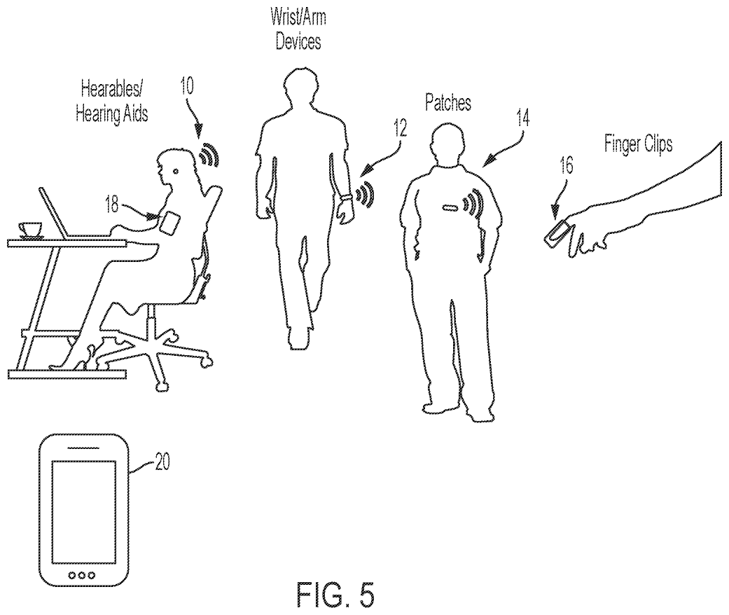

In some cases, the computational system may be worn as an ear-worn device (e.g., hearables/hearing aids) 10, as a limb-worn (e.g., wrist, arm, leg) device 12, as a patch 14, or as a finger clip 16, as illustrated in FIG. 5. Other form-factors, such as digit-worn devices (e.g., a finger or toe), apparel, etc., may alternatively comprise the computational system.

A key benefit of the biometric estimation (over the biometric measurement) is that the estimation may be continuous and painless, whereas the biometric measurement may be discrete and irksome to measure (such as measuring blood pressure with an automated cuff-based BP monitor or measuring blood glucose with a blood sample from a finger prick). Thus, even though the biometric estimation may have less measurement acuity than the actual biometric measurement, the ability to provide a “good enough” estimate (in between actual measurements) may outweigh the downside of potentially lower acuity.

These wearable PPG devices 12-16 may be in communication (e.g., electrical, optical, or wireless) with a blood pressure monitoring device, such as a blood pressure cuff 18 (such as that shown on the arm of the subject wearing the PPG earpiece 12 in FIG. 5). Alternatively, the blood pressure monitoring device may be another device. Just one of many additional examples would be a standoff device, such an electromagnetic wavelength doppler-based detection system or an imaging system (i.e., a camera). Other blood pressure monitoring devices may be used, as there are many known to those skilled in the art (ultrasound, arterial line, etc.). In another embodiment, the PPG measurements and BP measurements are received from the same device which is configured to measure both PPG and BP readings. One particular example of such a device comprises a cuff-based BP monitor having an integrated PPG sensor.

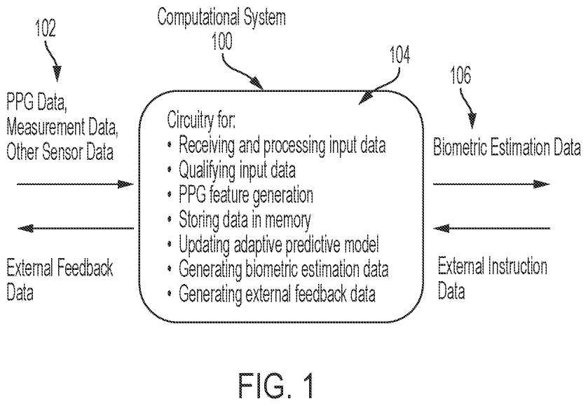

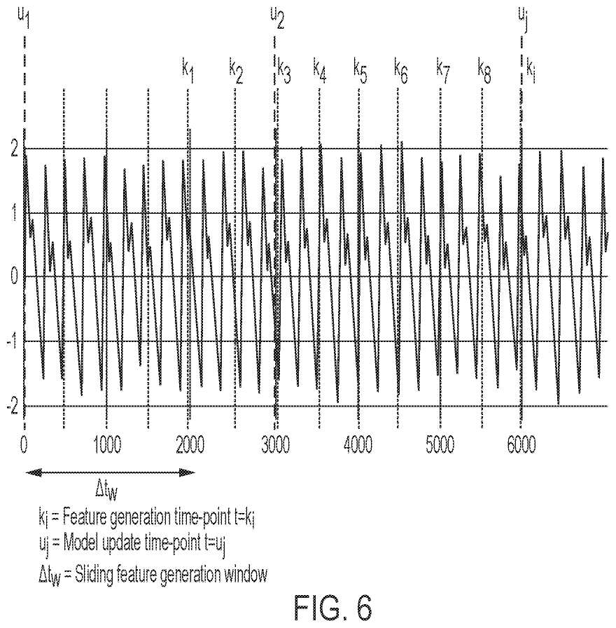

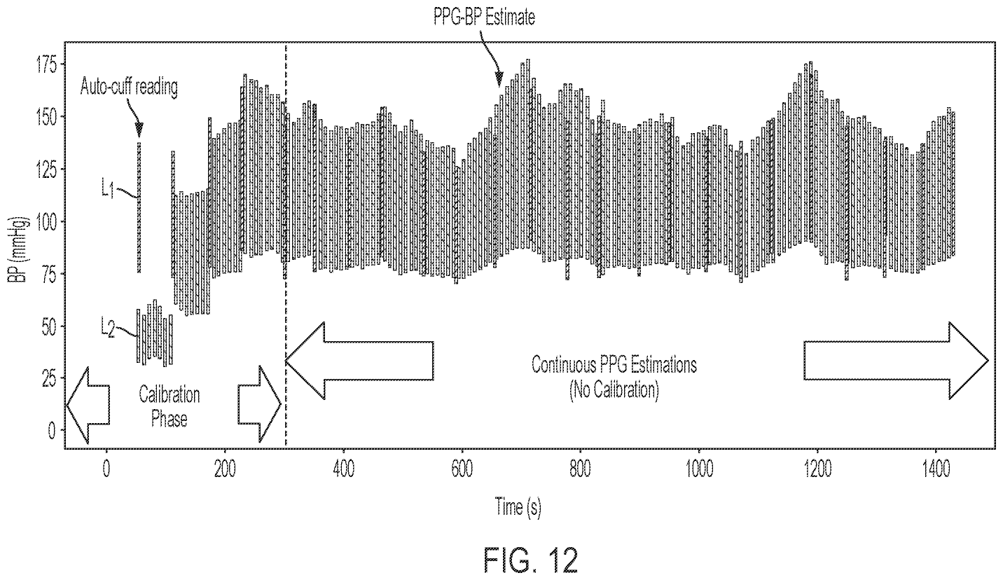

In some embodiments of the present invention, referred to as an adaptation process, a plurality of BP measurements from a cuff-based BP monitor 18 or other BP monitoring device and PPG measurements are processed together to improve the accuracy of the BP estimation. Once the model has been autonomously optimized for the subject and updated (FIG. 6), via a computational system (e.g., 100, FIG. 1) processing a plurality of BP measurements and PPG measurements collected as a temporal sequence, the blood pressure measurement device 18 (e.g., the cuff-based BP monitor) may no longer be needed, such that continuous PPG-based BP estimations may be generated in real-time via the updated model. In such case, this period of adaption may behave as a long-term calibration, which may be occasionally re-calibrated a few times of the day, week, month, or year with each new BP measurement (as shown in FIG. 12). In principle, as long as the relationship between one's PPG data and their BP does not change, and as long was the PPG data is collected in the same manner from the same location of the body, and as long as the BP estimation accuracy remains sufficient for the desired use case, then a single calibration may be sufficient indefinitely for the person.

Alternatively, BP measurements may be received and processed routinely, referred to as an augmentation process, such that the adaptive predictive model may be continuously augmented over time based on updated BP measurements (such as those taken from an automated cuff-based BP monitor). In augmentation, updating an adaptive predictive model according to embodiments of the present invention may be repeated continuously, several times an hour, with each new BP measurement update.

The triggering of a model update (FIG. 6) for either adaption or augmentation may be provided through a variety of methods. Examples of autonomous triggering paradigms may include, but are not limited to: 1) triggering based on a set timing protocol, 2) triggering based on motion sensing or activity status monitoring, 3) triggering based on the device being removed, re-worn, or relocated on the body, 4) triggering based on physiological status identification, and 5) triggering based on the detection of an error. Examples of triggering processing for wearables based on these paradigms have been previously presented in U.S. Pat. No. 9,538,921, which is incorporated herein by reference in its entirety. These autonomous triggers may be generated internally to the computational system 100 or externally (such as via and external device or via the external instruction data presented in FIG. 1). A key benefit of autonomous triggering is that substantial power savings can be realized if biometric measurements (such as cuff-based BP measurements) can be minimized while still retaining biometric estimation accuracy. Moreover, some biometric measurements, such as cuff-based BP measurements, can be burdensome to the user, and thus reducing the frequency of measurement while maintaining biometric estimation accuracy can be highly beneficial. In practice, the inventors have found that the computational power of wearable computers is quite sufficient for determining and executing such autonomous triggering.

Triggering based on a predetermined timing may be set as fixed or as a user adjustable parameter. This paradigm may be particular useful for hospital use cases and similarly immobile use cases, where the subject is lying at rest (e.g., in a hospital bed).

Triggering based on motion may be achieved by sensing activity above or below a threshold via a motion sensor (e.g., an accelerometer, imaging system, or other motion sensing device or component). This type of trigger may be especially useful for ambulatory monitoring of a biometric. If the motion status shows that excessive activity has occurred or that the frequency of excessive activity has increased, the computational system 100 may be triggered for more frequent model updates and the biometric measurement device may be triggered for more frequent measurements. This may help assure that estimation accuracy remains preserved. In keeping, if the motion status shows that the subject is resting or that the frequency of excessive activity is sufficiently low, the computational system 100 may be triggered for less frequent model updates and the biometric measurement device may be triggered for less frequent measurements. Similarly, this triggering may be dependent on activity status as opposed to a motion threshold. For example, an autonomously determined activity status of “running” or “walking” may trigger more frequent model updates and biometric measurements, whereas an autonomously determined activity status of “resting” or “sitting” may trigger less frequent model updates and biometric measurements. Methods of determining activity status via accelerometer data or imaging data are well known to those skilled in the art, such as in U.S. Pat. No. 10,610,158, which is incorporated herein by reference in its entirety.

If a wearable device according to embodiments of the present invention is removed from the subject and then re-worn, or if the device is repositioned on the subject, the biometric measurement device and the computational system may be triggered to take another biometric measurement and update the biometric estimation model, respectively. This autonomous triggering may help reestablish biometric estimation accuracy in the case that the wearable device has been temporarily disturbed or decoupled from the body. The autonomous determination that a device has been removed, repositioned, or re-worn can be executed by processing PPG data to determine signal quality or to determine other biometric parameters that may change as a wearable PPG device is positioned differently along the body. Methods of autonomously determining how a wearable device is being worn via PPG and motion sensing have been previously described, for example, in U.S. Pat. Nos. 9,794,653, 10,003,882, 10,512,403, and 10,893,835, the contents of which are incorporated herein by reference in their entireties.

A change in physiological status may also be autonomously detected and used to trigger another biometric measurement and update the biometric estimation model. For example, PPG sensor data (or other biometric data) may be processed to generate an assessment of stress status, cardiac status, respiratory status, or the like, and this physiological status update may be used as an autonomous trigger to take another BP measurement and update the model parameters. As one specific example, heart rate variability data from a PPG sensor (or other suitable biometric sensor) may be processed to indicate that one's stress state has changed (e.g., that the stress has notably increased or decreased), and this may provide the autonomous trigger. As another specific example, data from a PPG sensor (or other suitable biometric sensor) may be processed to generate subject breathing information (such as breathing rate, breathing volume, or breathing regularity (periodicity)/irregularity (aperiodicity)), and this may provide the autonomous trigger. For example, a significant change in breathing rate, breathing volume, or breathing regularity may be provide the autonomous trigger. This may be especially important for the accuracy of the invention in a field environment, as the transfer function between PPG information and subject blood pressure may be dependent on breathing dynamics. Methods of autonomously determining a change in physiological status via wearable PPG have been previously described, for example, in: U.S. Pat. Nos. 8,157,730, 8,929,966, 9,427,191, 10,413,250, 10,893,835, and 11,058,304, the contents of which are incorporated herein by reference in their entireties.

Similarly, in the case that an error (such as an operational error code) is detected by the computational system, an automatic triggering of a biometric measurement and model update may commence. This may help assure that accurate monitoring is robust to operational glitches. As one specific example, if the computational system receives an error code that the BP monitoring autocuff device which feeds the PPG-BP model has stopped inflating, this may trigger a system reset followed by another BP measurement and another PPG-BP model update.

Referring to FIG. 12, an example of an embodiment of the present invention utilizing real data collected from a human subject in a biometrics laboratory is illustrated. A human subject was wearing an automated BP cuff (at the brachial artery) and also wearing an ear PPG sensor, an arm (e.g., upper arm) PPG sensor, and a wrist PPG sensor (although only ear-PPG data is presented in FIG. 12 for simplicity). To compare the present invention to the volume clamp method, the subject was also wearing a volume-clamp device on the index finger of the arm where the BP cuff was located. The measurement sequence involved periods of subject rest followed by periods of subject activity. Namely, in order to increase the BP of the subject, the subject was asked to push against a stationary barrier with their legs for several seconds (an isometric leg press) while BP and PPG measurements were underway.

Then to decrease BP, the subject was asked to relax by terminating the isometric leg press. BP measurements from the cuff-based BP monitor (presented as a thick vertical line L₁, with the top point of the line L₁ representing the subject systolic BP and the bottom point of the line L₁ representing the subject diastolic BP) were received every 60-to-90 seconds and processed (by a computational system). During an initial calibration phase of approximately 300 seconds, multiple values from the cuff-based BP monitor were processed along with multiple PPG readings to generate multiple PPG estimates (presented as a thin vertical line L₂, in the same formalism as the cuff-based BP monitor readings). However, these estimates were not reported to the user, as the parameters of the adaptive predictive model were updated during this calibration phase to increase model accuracy such that it would be equivalent to that of the cuff-based BP monitor by the end of the calibration phase.

Following the calibration phase, continuous BP estimates were generated without updating model parameters for each new BP measurement. Rather, the remaining cuff-based BP monitor measurements are shown along with PPG estimations simply to note the excellent tracking between the PPG model estimates and the cuff-based BP monitor measurements. It should be noted that although the PPG estimates shown in FIG. 12 are from the ear PPG sensor only, it was discovered that equivalent performance can be realized via the wrist PPG sensor and the arm PPG sensor. However, for the case of the wrist PPG sensor and the arm PPG sensor, as the blood pressure cuff inflates and deflates, there is a period where occlusion can affect the blood flow (when the wrist and/or arm sensors are worn on the same arm as the cuff), such that meaningful PPG-BP estimations are not viable during the cuff-based BP monitor measurement period.

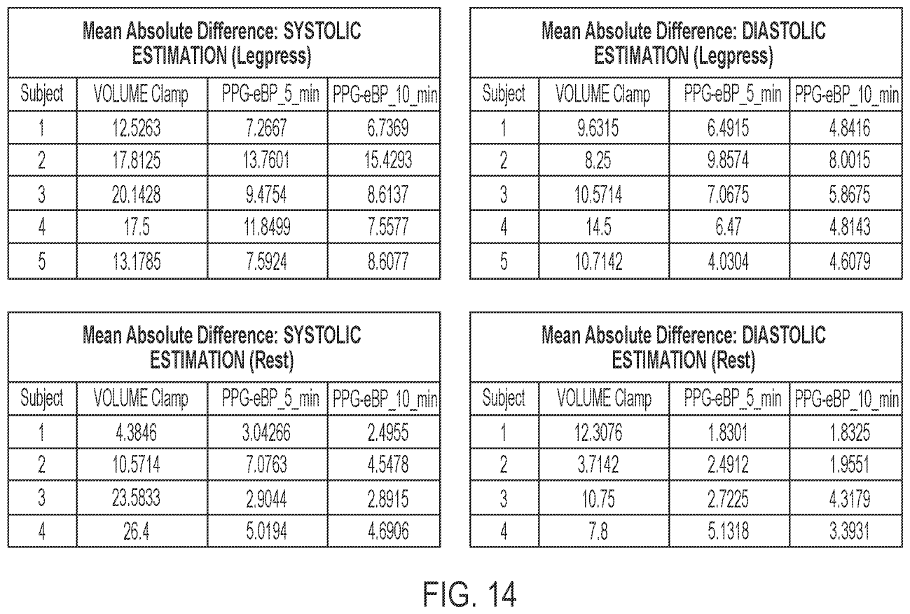

The test sequence of FIG. 12 was repeated on several subjects, and the performance of the PPG-BP estimation (also called the estimated BP measurement, or PPG-eBP) and the volume clamp device, as compared to the cuff-based BP monitor measurements, is presented in the tables of FIG. 14. As shown in FIG. 14, the mean absolute difference of the PPG-eBP is universally lower (better) than that of the volume clamp, both during the isometric leg press periods as well as the resting periods. It should be noted that, for each subject, a calibration period of both 5-minutes and 10-minutes was investigated, and a slight improvement in the PPG-BP model is observed for the longer calibration period (as can be derived from FIG. 14).

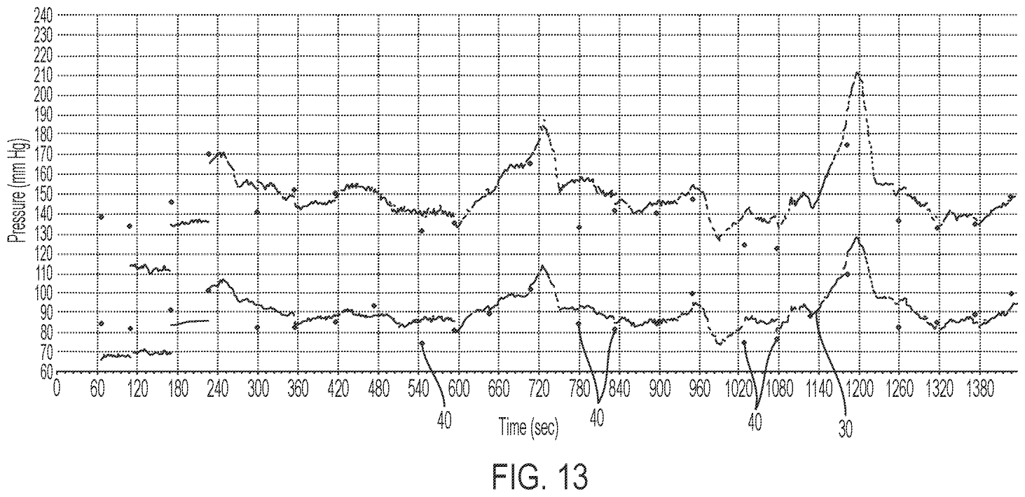

Referring to FIG. 13, BP estimates for a subject wearing a PPG sensor made over time via an adaptive predictive model in accordance with embodiments of the present invention are illustrated and represented by the plot 30. Actual blood pressure measurements (readings) from a monitor attached to the subject are represented by the data points 40. BP estimation accuracy is improved over time as the adaptive predictive model is updated with each BP measurement 40, and this is illustrated in FIG. 13 as the differential between the plot 30 and the data points 40 decreases over time. In FIG. 13, the PPG-BP estimation plot 30 is shown with a second-by-second estimation frequency. However, the estimation frequency need not be fixed in this invention, and the resolution of this BP estimation may be decreased or increased depending on the use case (e.g., depending on the BP estimation accuracy or resolution requirements of the use case), the accuracy and resolution of the benchmark BP measurement device, and the feature generation frequency selected for the adaptive predictive model (FIG. 6). For example, an estimated BP pulse wave trace (i.e., a complete “beat-to-beat” BP waveform having a resolution much less than 1 second) may be generated via this invention, as long as the BP measurement device has sufficient accuracy and resolution to sufficiently train the adaptive predictive PPG-BP model.

It should be emphasized that this invention is not limited to PPG-based BP estimations but may also be applied towards other PPG-based biometric estimations. Moreover, other measurement modalities outside of BP measurements may be used as the benchmark and basis for updating the adaptive predictive model. Nonlimiting examples of such biometric measurements and respective biometric estimations may comprise measurements and estimates of: breathing (respiration) rate, heart rate, cognitive load, intent (e.g., to take a mental or physical action), cardiac output, cardiopulmonary functioning, a cardiac condition or disease state (such as an arrythmia, premature contraction of the heart, heart damage, heart disease, plaque build-up, and the like), gas-exchange dynamics, blood analyte constituents (e.g., blood glucose level, blood urea level, bilirubin level, cholesterol level, etc.), and the like. Additional examples of other measurement/estimation modalities include monitoring ECG, EEG, EMG, EOG, blood flow volume, chest impedance, auscultatory monitoring, arterial line data, bloodwork data, and the like. As a specific example, it may be desirable to monitor the EEG readings for a subject to study brain wave patterns during an activity or during sleep. However, EEG is notoriously uncomfortable to wear, especially for sleep, where it would be more desirable to monitoring one's EEG with a more comfortable technology, such as PPG. Thus, a set of EEG electrodes may be used to provide EEG measurement data to be fed into the adaptive predictive model, and a different sensor modality, such as PPG, may be monitored simultaneously and fed into the adaptive predictive model to create a model for estimating EEG via PPG data (without requiring EEG data). Namely, once the PPG model has adapted to the EEG data, estimating the real-time EEG for a subject may then commence using real-time PPG data alone (i.e., without EEG sensing required).

### Method of Generating a Biometric Estimation for a Subject Via an Adaptive Predictive Model

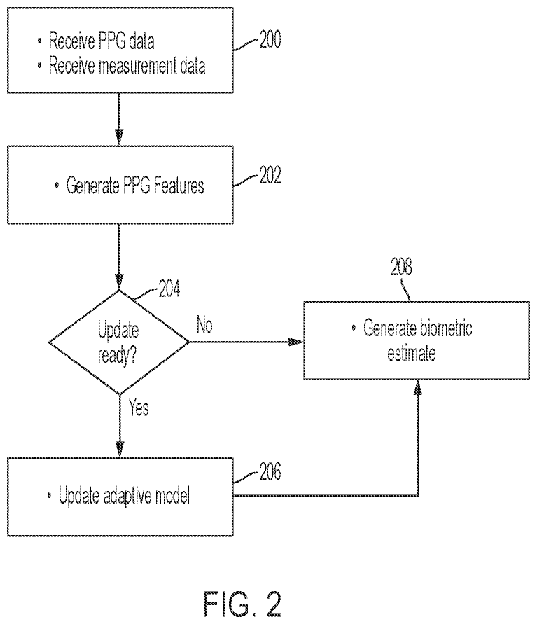

Referring to FIG. 2, a method of generating a biometric estimation for a subject according to some embodiments of the present invention, via a real-time adaptive predictive model executed via a computational system is illustrated. The method includes receiving, within a receiving period, real-time PPG data from a PPG sensor configured to measure PPG information from a subject, and receiving, within the receiving period, a real-time blood pressure measurement from a blood pressure monitoring device configured to measure the blood pressure of the subject (Block 200). Features are generated from the received PPG data (Block 202). The generated features and the blood pressure measurement are stored in memory. If an update is ready (Block 204), the adaptive predictive model may be updated in real-time by processing the stored features and the stored blood pressure measurement to generate updated model parameters that reduce the estimation error of the adaptive predictive model (Block 206). A biometric estimation for the subject is then generated via the updated adaptive predictive model (Block 208).

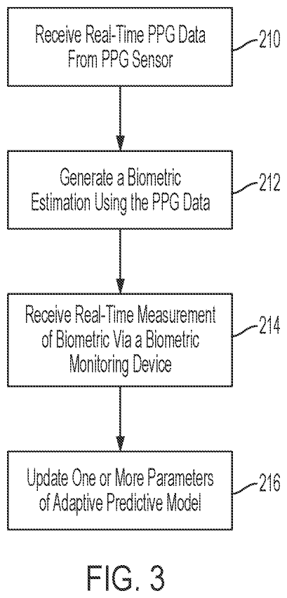

Referring to FIG. 3, a method of generating a biometric estimation for a subject, according to other embodiments of the present invention, is illustrated. Real-time PPG data is received by a computational system (e.g., 100, FIG. 1) from a PPG sensor (e.g., 12-16, FIG. 5) attached to a subject (Block 210). The computational system generates a biometric estimation for the subject via an adaptive predictive model using the PPG data (Block 212). An exemplary biometric estimation is a blood pressure estimation for the subject, although various other biometrics may be estimated, as will be described later. A real-time measurement of the biometric from a monitoring device (e.g., a blood pressure cuff 18, FIG. 5) attached to the subject is received by the computational system (Block 214) and the computational system updates one or more parameters of the adaptive predictive model (Block 216). For example, a real-time blood pressure reading is obtained from the subject via a blood pressure monitoring device and this reading is used to adjust the adaptive predictive model such that the blood pressure estimation made by the adaptive predictive model using PPG data is closer to the actual blood pressure reading.

It is to be understood that the steps illustrated in FIG. 3 need not occur in the illustrated order. For example, real-time biometric measurements (Block 214) may be collected prior to, or in unison with, the real-time PPG data collection (Block 210).

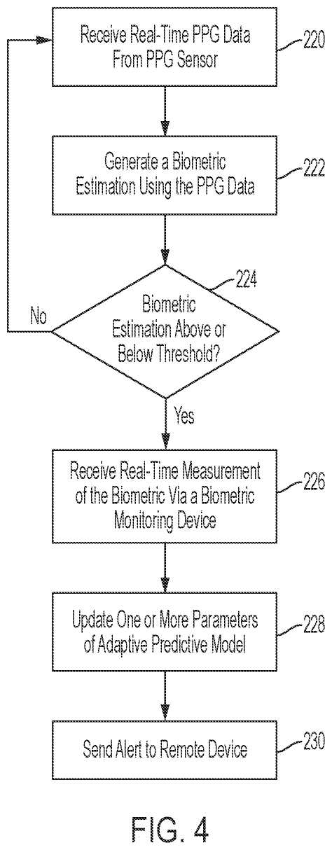

Referring to FIG. 4, a method of generating a biometric estimation for a subject, according to other embodiments of the present invention, is illustrated. Real-time PPG data is received by a computational system (e.g., 100, FIG. 1) from a PPG sensor (e.g., 12-16, FIG. 5) attached to a subject (Block 220). The computational system generates a biometric estimation for the subject via an adaptive predictive model using the PPG data (Block 222). An exemplary biometric estimation is a blood pressure estimation for the subject, although various other biometrics may be estimated, as will be described later. A determination is made whether the biometric estimation is above or below a threshold (Block 224). For example, a healthy blood pressure range is typically considered as systolic blood pressure less than 120 mmHg and diastolic less than 80 mmHg. However, if systolic blood pressure drops below 90 mmHg and/or diastolic blood pressure drops below 60 mmHg for a subject, medical intervention may be necessary. Similarly, if systolic blood pressure rises above 130 mmHg and/or diastolic blood pressure rises above 90 mmHg, medical intervention may be necessary.

If the biometric estimation is above or below a threshold (Block 224), a real-time measurement of the biometric is received by the computational system from a biometric monitoring device (e.g., a blood pressure cuff 18, FIG. 5) attached to the subject (Block 226) and the computational system updates one or more parameters of the adaptive predictive model (Block 228). For example, a real-time blood pressure reading is obtained from the subject via a blood pressure monitoring device and this reading is used to adjust the adaptive predictive model such that the blood pressure estimation made by the adaptive predictive model using PPG data is closer to the actual blood pressure reading. In addition, the computational system sends an alert to a remote device that the biometric estimation is above or below a threshold (Block 230).

It should be noted that BP estimation does not have to fall outside of a range in order for a calibration cuff reading to be called for and then used to increase accuracy of the estimations. Estimated BP can be in a normal range and a subsequent cuff reading can still be used to refine the accuracy. The adaptive predictive model can be updated merely based on set timed cuff-based readings, without regard to BP values versus a threshold. Similarly, the adaptive predictive model may be updated due to sensed changes in activity level (e.g., sensing a change in body motion via an accelerometer) or due to other sensor readings.

A remote device may be a smartphone of a medical provider, a nurse's station in a medical facility, or any other device that can alert a medical person as to the condition of the subject. The alert may also be sent to the blood pressure monitoring device (e.g., the blood pressure cuff 18, FIG. 5). In addition, the alert could be generated by the blood pressure monitoring device.

It should be noted that, although Block 224 is presented as a “threshold” decision, Block 224 may be replaced with conditional logic for determining that a model update should occur. For example, rather than threshold logic based on one biometric estimation, Block 224 may comprise logic to determine the existence of a thresholding pattern of a plurality of biometric estimations (e.g., that have been stored in memory). In one nonlimiting example, this pattern may comprise a series of consecutively high (above normal) or consecutively low (below normal) BP estimations, and the determination of this pattern may then trigger a model update. In another nonlimiting example, this pattern may comprise an average of multiple biometric estimations that is determined to be above or below normal; if the pattern is determined to exist, then a model update may be triggered. The methods illustrated in FIGS. 2-4 may be executed via a computational system 100, such as that shown in FIG. 1. The computational system 100 may comprise: 1) at least one data bus 102 for receiving PPG data from a PPG sensor configured to measure PPG information from the subject (as well as optional additional sensor data, such as, but not limited to, motion sensor data or environmental sensor data from supportive sensors, such as, but not limited to, motion sensors, environmental sensors, etc.) and blood pressure data from a blood pressure monitoring device configured to measure a blood pressure of the subject and 2) computational circuitry and instructions 104 configured to receive, within a receiving period, PPG data from the PPG sensor; receive, within the receiving period, a blood pressure measurement from the blood pressure monitoring device (such as an automated blood pressure cuff, arterial line measurement, etc.); generate features from the received PPG data; store the features in memory; store the blood pressure measurement in memory; update the current parameters of the adaptive predictive model by processing the stored features and the stored blood pressure measurement to generate updated model parameters that reduce the estimation error of the adaptive predictive model; and generate a biometric estimation for the subject by executing the updated adaptive predictive model.

### Receive PPG Data; Receive BP Measurement

Referring back to FIG. 2, updating an adaptive predictive model (Block 206) requires at least two inputs: PPG features and at least one BP measurement. The data may be received over a “receiving period”, referring to a period of time wherein at least one PPG waveform and at least one time-correlated BP measurement has been received by the computational system 100 of FIG. 1. The received PPG data may be received as digitized data, and thus a prior digitization step may be required to digitally sample the PPG data (e.g., at a frequency of “fₛ”) before it is received by the computational system 100 of FIG. 1. The BP data may be received digitally as well, and thus a prior digitization step may also be required. However, discrete BP values may be received rather than streaming continuous BP values due to the discrete nature of cuff-based BP measurements. Although the PPG data and the BP measurement must be time-correlated (sufficiently close together in time), these measurements do not need to be exactly time-coincident (occurring exactly at the same time). This is because BP may not change dramatically over the course of a few seconds in the vast majority of circumstances, and during these few seconds several PPG waveforms may be received. Moreover, because PPG data may be continuously collected, whereas cuff-based BP measurements may require more than 60-to-90 seconds in between measurements, it may be impractical to perfectly align each PPG waveform with a coincident BP waveform (or BP measurement). For a typical ambulatory resting state, a time-correlation between the PPG data and BP measurements within ˜30 seconds has been shown to be sufficient for continuous tracking. This timing may be longer or shorter depending on the activity status of the subject, the dynamics of the subject's cardiac output, or other factors that may affect the rate of BP changes or other physiological changes for the subject. This time-correlated PPG and BP measurement data may be stored in memory (such as a memory buffer) via the computational system.

### Generate PPG Features

The received PPG data is processed to generate a plurality of real-time PPG features (Block 202, FIG. 2). Each of these features may be a characteristic feature that is distinct from the other features, for a total of “n” characteristic features. Exemplary features include, but are not limited to time-domain features or transform-based features. Nonlimiting examples of time-domain features may comprise PPG amplitude, PPG upper and/or lower envelope, systolic and diastolic peak separation and/or relative amplitude, systolic and dicrotic notch peak-to-trough separation, temporal separations between key features (such as peaks or troughs) in a PPG waveform, and the like. Similarly, the PPG data may be processed to generate a derivative (e.g., a 1^{st}, 2^{nd}, 3^{rd}, etc. derivative) or an integral, and time-domain features of these derivative and/or integral waveforms may be generated (i.e., generating features for amplitude, relative amplitude of peaks or troughs, upper and/or lower envelope, temporal peak separations, and the like). Transform-based features may comprise spectral features, wavelet features, the Teager-Kaiser energy (KTE) operator based features, chirplet transform features, noiselet transform features, spaceogram features, shapelet features, derivative features, integral features, principle component analysis (PCA) features, and the like.

Generating features from the received PPG data may comprise generating features at feature generation intervals (time-points) t=kᵢ within the receiving period via a sliding window of time Δt_{w} (FIG. 6). Features may be generated at any point in time by the computational system; however, enough PPG data must be stored in memory in order to process a meaningful PPG feature—at least one full PPG wave, and preferably a plurality of PPG waveforms. For example, features may be generated at t=kᵢ over a feature generation window, by processing buffered digitized PPG data collected over a prior period of time that is Δt_{w} long (i.e., a window of time Δt_{w} wide). This feature generation window may comprise a sliding window, such as a FIFO (first-in-first-out) buffer, wherein the PPG data is stored in the buffer, continuously gaining a new sample of data, and losing the oldest sample of data over time. The feature generation process may comprise processing this buffered PPG in the time domain or via a transform of the stored time-domain data. As noted above, a variety of different time-domain or transform-based processing methods may be utilized for generating the PPG features. Non-limiting examples of transforms for generating PPG features may comprise: spectral transforms, wavelet transforms, the Teager-Kaiser energy operator, chirplet transforms, noiselet transforms, spaceograms, shaplets, derivatives, integrals, and the like. Nonlimiting examples of time-domain processing may comprise processing steps for generating: PPG amplitude, PPG upper and/or lower envelope, systolic and diastolic peak separation, systolic and dicrotic notch peak-to-trough separation, and the like. Nonlimiting examples of transforms and time-domain processing that may be utilized are presented in U.S. Pat. No. 10,856,813 and PCT Application No. US20/49229, which are incorporated herein by reference in their entireties.

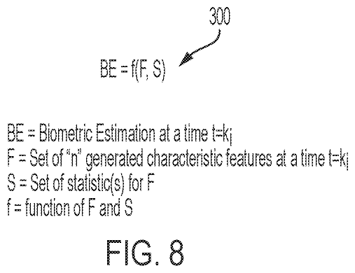

It should also be noted that, prior to generating a BP estimate (or other biometric estimate), the PPG features (characteristic features) may be actively normalized (e.g., weighted), to help ensure smooth temporal tracking of PPG-based BP estimations (or other biometric estimations) with BP measurements (or other biometric measurements). One normalization approach is to process the statistics of the stored features (e.g., the prior stored PPG features in memory) and to normalize by these statistics. Normalization may be performed by processing historical data over a plurality of feature generation time-points, by generating statistics for the historical data and normalizing by these statistics. This normalization process may be updated with each new feature generation time point (e.g., t=kᵢ of FIG. 6 and FIG. 8). Alternatively, normalization may be performed with each model update (e.g., t=uⱼ of FIG. 6). There are numerous normalization methodologies known to those skilled in the art; a few examples may comprise: z-norming, min-max normalization, mean normalization, and the like. One nonlimiting normalization method is to employ Cochrane's equations for pooled statistics. To employ Cochrane's equations with each model update, the mean and standard deviation of each characteristic feature may be normalized (weighted) by processing (pooling) the statistics of the features from the past update (e.g., at t=uⱼ₋₁) with the statistics of the features following the past update (e.g., at t=uⱼ). Thus, the pooled mean and standard deviation generated by Cochrane's equations may be utilized as the basis for normalizing the characteristic features. As a specific non-limiting example, utilizing the z-norming method, the value of characteristic features may be normalized by the mean and standard deviation generated by Cochrane's equations—e.g., wherein this mean and standard deviation is generated by weighting the mean and standard deviation for the features from the past update (e.g., at t=uⱼ₋₁) with the mean and standard deviation of the features following the past update (e.g., at t=uⱼ).

The aforementioned feature statistics themselves may also be employed as features to an adaptive predictive model, according to embodiments of the present invention. This may help provide smoother tracking (e.g., of BP estimations vs. BP measurements).

It should be noted that, as part of (or prior to) feature generation, preprocessing of the received sensor information (e.g., the PPG sensor data) and/or the received biometric measurement data (e.g., the BP measurement data) may be required. Additionally, it may be important to qualify the received data to reject “bad” data, generate a confidence score for the data, identify “good” data, or to classify data for further processing. A variety of preprocessing methodologies for PPG data (including associated motion sensor data) have been previously published and are well known to those skilled in the art, including, but not limited to: U.S. Pat. Nos. 10,834,483, 10,798,471, 10,631,740, 10,542,893, 10,512,403, 10,448,840, 9,993,204, 10,413,250, and PCT Application No. US20/49229, all of which are incorporated herein by reference in their entireties. Both passive and active methodologies of removing subject motion noise may be employed. Moreover, it should be noted that the optimal preprocessing may be feature-dependent. For example, regarding PPG data, for spectral domain features it may be desirable to remove or attenuate the “DC component” (e.g., the non-pulsatile component) from the PPG signal before feature generation. However, the DC component may be important for other features (such as time-domain features), or the DC component may even be a feature in itself. It should also be noted that PPG sensor data may comprise subject motion data (as described earlier), and this motion data may be utilized to reduce motion artifacts from optical sensor readings. The motion sensor may be integral to, or collocated with, the PPG sensor. Motion sensor data may be processed as a feature as well.

Preprocessing of biometric measurement data may also be useful. For example, in a preferred use case, a BP measurement from a BP cuff may comprise a discrete value of systolic and diastolic BP measurements. In some use cases, this data may be available to the computational system through an API (application programming interface) or through an application-specific interface. However, in some use cases, the BP measurement data received by the computational system of FIG. 1 may comprise a data stream (such as a raw data stream) where the BP measurement may need to be extracted via processing before the invention may be executed.

### Update Model Parameters

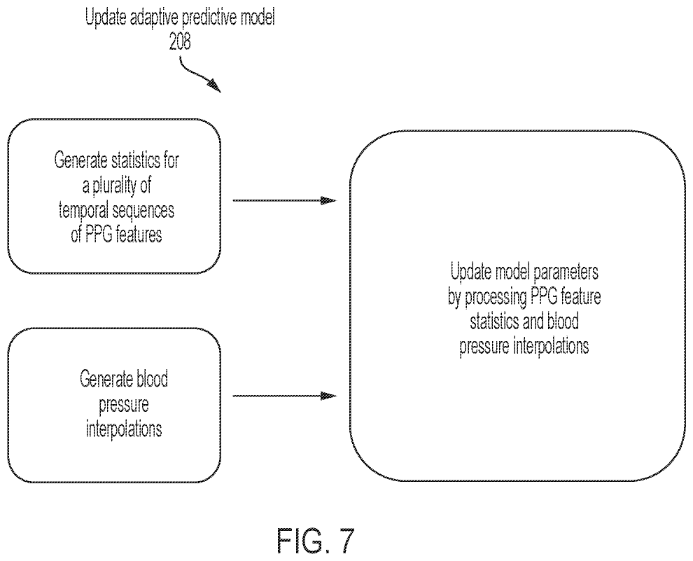

Referring to FIG. 8, an adaptive predictive model 300 for generating a biometric estimation (BE) may take the form of BE=f(F, S), where F is a set of “n” generated characteristic features (e.g., normalized features) at a time t=kᵢ, and where S is a set of statistic(s) for F. The function f(F,S) may comprise a transfer function connecting the biometric estimation with the aforementioned features and statistics. For each new BP measurement (or biometric measurement) received, the adaptive predictive model 300 may be updated (as shown in FIG. 7) at each new update time-point t=uⱼ. Updating the model comprises updating one or more parameters of the adaptive predictive model 300.

Depending on the type of model used, the model parameters may be different. For example, in a regression model, the model parameters may comprise at least one coefficient to the regression model. Nonlimiting examples of suitable regression models may comprise: linear, SVM, random forest, neural network, decision trees, a combination of these models, and the like. Other types of models outside of regression models may also be utilized; as a nonlimiting example, a classifier may be utilized, or a combination of classification and regression (as may be utilized in a convolutional neural network (CNN)). Updating the model may comprise processing the characteristic features (e.g., normalized characteristic features) and a stored blood pressure measurement to generate updated model parameters that reduce the estimation error of the adaptive predictive model 300. For example, the regression model may be solved for the recent BP measurement (or biometric measurement) and then the model coefficients may be updated. Alternatively, or additionally, a gradient-based optimization approach may be employed (such as classical gradient descent, Adam, Momentum, AdaGrad, RMSProp, AMSgrad, or the like) to update model coefficients with each new BP measurement.

Updating the adaptive predictive model in real-time may comprise processing a recent stored blood pressure measurement (associated with timepoint t=uⱼ) and a prior stored blood pressure measurement (associated with time-point t=uⱼ₋₁). In one embodiment, this may comprise generating an interpolation of expected blood pressure measurements (i.e., a temporal interpolation) between blood pressure measurements collected over time, such as an interpolation between the recent stored blood pressure measurement and the prior stored blood pressure measurement (or a plurality of prior stored blood pressure measurements). A specific example can be summarized in context of FIG. 6. A blood pressure measurement associated with time-point u₂ and a blood pressure measurement associated with time-point uⱼ (in this particular case u₃) may be stored in memory and processed to generate an interpolation of expected blood pressure measurements for plurality of feature generation intervals, such as for each feature generation interval t=kᵢ. In such case, updating the adaptive model may then comprise updating the model parameters in context of each feature set and each interpolated BP measurement over a plurality of intervals t=kᵢ. Thus, there is more information by which to optimize the regression model than just 2 blood pressure measurements, leading to smoother tracking of the BP estimation with the actual BP measurements.

### Generate Biometric (BP) Estimation

As summarized above, there are many model constructs that may be used to generate the biometric estimation, and the general formalism of the function used to generate the biometric estimation is presented in FIG. 8. For the specific case that has been described with respect to generating blood pressure estimations, the process of generating a BP estimation may comprise generating a systolic blood pressure, a diastolic blood pressure, a pulse pressure, a mean arterial pressure, or another type of pressure associated with blood flow. Moreover, the type of blood pressure that may be estimated may from virtually any location on the body, such as (but not limited to) brachial, thoracic, subclavian, femoral, tibial, radial, carotid, central (aortic), cerebral, or the like. Each of these blood pressure estimations may be generated using the methods of FIGS. 2-4, via the processes summarized above; however, the BP measurement locations on the subject should ideally match that of the desired BP estimations. Namely, if the desired biometric estimation comprises systolic and diastolic estimations of the brachial artery, then the BP monitoring device providing the BP measurements should (ideally) measure both the systolic and diastolic BP values from the brachial artery.

### Computational System for Generating a Biometric Estimation Via an Adaptive Predictive Model

For implementing the methods of FIGS. 2-4 a computational system 100 may be utilized, as shown in FIG. 1. The computational system 100 for generating a biometric estimation (in this particular case a BP estimation) for a subject via an adaptive predictive model may comprise: 1) at least one data bus 102 for receiving PPG data from a PPG sensor configured to measure PPG information from the subject and blood pressure data from a blood pressure monitoring device configured to measure a blood pressure of the subject and 2) computational circuitry and instructions 104 configured to: a) receive, within a receiving period, PPG data from the PPG sensor, b) receive, within the receiving period, a blood pressure measurement from the blood pressure monitoring device, c) generate features from the received PPG data, d) store the features in memory, e) store the blood pressure measurement in memory, f) update the current parameters of the adaptive predictive model by processing the stored features and the stored blood pressure measurement to generate updated model parameters that reduce the estimation error of the adaptive predictive model, and g) generate a biometric estimation for the subject by executing the updated adaptive predictive model.

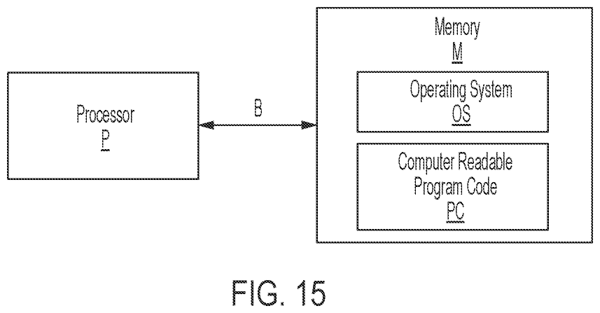

The computational system 100 may be implemented as a plurality of discrete components, a fully integrated system, or a mixture of both. As a specific example, the computational system 100 may comprise a fully integrated microprocessor, with computational instructions for executing the processing steps of FIGS. 2-4. FIG. 15 is a block diagram that illustrates details of an example processor P and memory M that may be used in accordance with various embodiments of the present invention. The processor P communicates with the memory M via an address/data bus B. The processor P may be, for example, a commercially available or custom microprocessor. Moreover, the processor P may include multiple processors. The memory M may be a non-transitory computer readable storage medium and may be representative of the overall hierarchy of memory devices containing the software and data used to implement the methods of FIGS. 2-4 as described herein. The memory M may include, but is not limited to, the following types of devices: cache, ROM, PROM, EPROM, EEPROM, flash, Static RAM (SRAM), and/or Dynamic RAM (DRAM).

The memory M may hold various categories of software and data, such as computer readable program code PC and/or an operating system OS. The operating system OS may control operations of the processor P, a PPG sensor (e.g., 12-16, FIG. 5), a biometric monitoring device (e.g., BP cuff 18, FIG. 5) and may coordinate execution of various programs by the processor P. For example, the computer readable program code PC, when executed by a processor P, may cause the processor P to perform any of the operations illustrated in the flowcharts of FIGS. 2-4.

Alternatively, the computational system 100 may comprise an analog circuit configured to process the steps through analog processes. As another example, the computational instructions may be executed as a software library executed via a computational system (such as a processor). As another example, the computational system may comprise neural circuitry or quantum computing. Traditional, quantum, or neural processors may be utilized, or a combination of each.

A variety of components for enabling the system 100 of FIG. 1 are well known to those skilled in the art. The computational resources required to execute the methods of FIGS. 2-4 via a microprocessor are practical for a wearable or portable system, as the inventors have demonstrated via laboratory testing that suitable real-time performance can be achieved utilizing computational instructions (algorithms) executed via software on a commercially available smartphone 20 in communication with a wearable device 10-16, as illustrated in FIG. 5.

The system may comprise input/output lines (i.e., ports or buses) to communicate with other systems, for receiving and giving data from/to external systems. For example, the input/output lines may communicate with at least one external processor, computational system, or apparatus. In one specific embodiment, a biometric estimation generated may be digitized and made available to an external computational system via a digital bus 106. In another embodiment, the input/output lines may communicate with one or more transceivers for communicating wirelessly with an external system. A variety of electronic communication configurations are well known to those skilled in the art.

In the case where a BP estimation is generated by the computational system of FIG. 1, for use by an external system, the external system may wish to send information to the computational system for modifying a computational process (i.e., modifying algorithms). For example, in one embodiment, the BP estimation generated may comprise a brachial BP estimation, where a remote system (in wired or wireless communication with the computational system) may comprise a BP cuff that feeds BP measurements to the computational system of FIG. 1. The BP cuff may also comprise a viewing screen to view PPG-BP estimation readings, generated by the computational system, in between BP measurements. It may be desirable to change the rate of PPG processing (such as the sampling rate, feature generation interval, update interval, or the like) via an interface on the BP cuff, and this information may then be fed to the computational system of FIG. 1 as “external instruction data” for executing this desired change. Alternatively, or additionally, the computational system may have feedback to provide the external system (i.e., the BP cuff), such as warnings that sensor estimations may be inaccurate due to motion noise, or other useful information. Similarly, either the computational system or the external system may provide information regarding when the BP measurements and/or BP estimations should commence (e.g., the frequency of BP measurements and/or BP estimations).

It should be noted that one form of external system data may comprise meta data for the subject, and this meta data may be useful in processing biometric estimations in accordance with embodiments of the present invention. Namely, the computational system 100 of FIG. 1 may receive external meta data (i.e., height, weight, age, sex, medication usages, and the like) for the subject and store this data in memory. The meta data may be utilized as a feature to the adaptive model 300 of FIG. 8. Alternatively, or additionally, this stored meta data may be utilized to create a profile for the subject. The profile may comprise parameters for the adaptive model that have been optimized for the subject (i.e., over the course of several biometric measurements). A key benefit of a user profile is that it may prevent model adaption delays caused by a “cold start” (i.e., the subject starting a new estimation/measurement session). Phrased another way, a finite period of time may be required to adapt (calibrate) to the subject (as shown in FIG. 12), and this calibration phase can be shortened if the previous model parameters for the subject have been stored in memory.

### Other Biometric Estimations

As noted earlier, the system of FIG. 1 and the corresponding methods of FIGS. 2-4 (as well as the supplemental examples of FIGS. 6, 7, and 8) may be applied more broadly than for a continuous estimation of BP. A variety of continuous physiological estimations may be realized via embodiments of the present invention. Namely, other than the biometric estimates being generated and the biometric measurements being received, the other core elements of the invention of FIG. 1 and FIGS. 2-4 may remain in place.

Because PPG information comprises rich information regarding blood flow, blood pressure is just one of many real-time hemodynamic parameters that may be extracted via embodiments of the present invention. Namely, a variety of hemodynamic parameters may be estimated via embodiments of the present invention, such as (but not limited to): arterial blood pressure, mean arterial pressure, systolic pressure variation, pulse pressure variation, stroke volume variation, right arterial pressure, right ventricular pressure, pulmonary artery pressure, mean pulmonary artery pressure, pulmonary artery wedge pressure, left atrial pressure, cardiac output, cardiac index, stroke volume, stroke volume index, systemic vascular resistance, systemic vascular resistance index, pulmonary vascular resistance, pulmonary vascular resistance index, stroke work index, ejection fraction, and the like. The ability of embodiments of the present invention to estimate these parameters is contingent on the right measurement device. For example, accurately estimating real-time ejection fraction via embodiments of the present invention would require collecting time-correlated measurement data form an accurate benchmark device, such as an echocardiogram monitoring device.

As just one example, measurement data may comprise EEG measurements and the associated biometric estimation that is generated may be an estimation of an EEG assessment. Nonlimiting examples of EEG assessments may comprise alertness level, dominating pattern (e.g., alpha, beta, theta, or delta), subject intent, identification of an abnormality, identification of normality, brain disorder, sleepiness, wakefulness, or the like. In this case, at least some of the characteristic features generated from processing the PPG sensor would be given significantly more or less weight than those used in estimating blood pressure. This is because the physiological relationship between EEG and PPG is quite different than that of BP and PPG. For example, PPG features related to time-domain variations in PPG peak locations (such as heart rate variability, the temporal location of systolic and diastolic peaks, as well as the temporal location of the dicrotic notch) may be more tightly correlated with EEG features.

As another example, if the measurement data comprises a gas-exchange (respiratory) analysis measurement, the associated biometric estimation that is generated may comprise an estimation of a gas-exchange analysis measurement. Nonlimiting examples of gas-exchange analysis measurements may comprise: carbon dioxide, oxygen, arteriovenous oxygen difference, exercise oscillatory breathing, fraction of carbon dioxide or oxygen in expired air, expiratory volume, metabolic equivalent, maximum voluntary ventilation, oxygen-uptake efficiency slope, partial pressure of end-tidal carbon dioxide or end-tidal oxygen, carbon dioxide output, respiratory exchange ratio, minute ventilation, volume of dead space, ventilatory threshold, ventilatory equivalent of oxygen or carbon diode, oxygen update, or the like.

In a similar example, if the measurement data comprises arterial blood gas measurements, the associated biometric estimation that is generated may comprise an estimation of an arterial blood gas measurement. Nonlimiting examples of arterial blood gas measurements include: H+ or pH level, total CO₂, O₂ content, partial pressure of oxygen or carbon dioxide, HCO₃⁻ (bicarbonate), SBCₑ, base excess, arterial oxygenation, venous oxygenation, oxygen extraction, or the like. For the cases of generating a gas-exchange analysis estimation or an arterial blood gas estimation, it may be especially important to receive PPG data comprising simultaneous multiwavelength (MWL) data (such as streaming PPG data from a MWL PPG sensor), in order to generate a set of characteristic features for a plurality of wavelengths of light. This is because the PPG feature distribution may be dependent upon the wavelength of light used, and this distribution may modulate differently in time depending on the respiratory or blood gas parameter being monitored. As just one example, the relative amplitude of PPG signals for different wavelengths of light will modulate in a characteristic way for low vs. high levels of blood oxygenation.

In another example, if the measurement data of FIG. 1 and FIGS. 2-4 comprises a core body temperature measurement, the associated biometric estimation that is generated may comprise an estimation of core body temperature. It is known that PPG information, particularly heart rate changes, are correlated with core body temperature (see, for example, U.S. Pat. No. 10,206,627, which is incorporated herein by reference in its entirety), and thus characteristic PPG features exist to map subject PPG data with temperature measurements. Because it is difficult to measure core body temperature continuously throughout daily life activities, the invention herein enables a beneficial method of estimating core body temperature in an ambulatory fashion via processing data collected from a PPG device based on an adaptive model that has been previously updated by core body temperature measurements.

In another example, if the measurement data of FIG. 1 and FIGS. 2-4 comprises blood glucose levels, the associated biometric estimation that is generated may comprise an estimation of blood glucose. In this use case, the subject may be wearing a glucometer (such as a continuous glucose monitor—CGM) or routinely pricking their fingers to generate a series of blood glucose measurements. These glucose measurements may be received by the computational system 100 (FIG. 1), along with streaming PPG data, to process the combined data and generate a PPG-based glucose estimation, according to embodiments of the present invention. As summarized in PCT Application No. US20/49229, which is incorporated herein by reference in its entirety, there are PPG features that are correlated with blood glucose trending, particularly features associated with respiration-related changes and arterial compliance changes expressed within multiwavelength PPG data. These features may serve the basis of the features for FIG. 8, and the statistics for updating model parameters may be statistics of these respiration-related or arterial compliance-related features (or other features). Once the calibration phase following multiple glucose measurements is completed, and the adaptive model is stabilized (i.e., with no model updates required) for continuous PPG-based glucose estimations, the subject may be liberated from invasive (or minimally invasive) blood glucose measurements for an extended duration of time. With an extended duration of time (such as several hours, days, or weeks) before an additional glucose measurement is provided to the adaptive predictive model, the precision in glucose estimations via PPG during this extended duration may be lower than that which would be provided by glucose measurements from a CGM or finger prick. But the benefit of the painless PPG-based glucose estimations may outweigh the reduced precision in many use cases. For example, the PPG estimation approach (once calibrated to the glucose measurements) may be particularly useful for predicting or estimating a steep rise or steep fall in blood glucose levels so that the subject may be notified to collect a glucose measurement (i.e., to take a blood sample or other fluid sample) to more precisely confirm blood glucose status.

### Imaging Applications

As noted earlier, one type of PPG sensor that may be utilized in the system and methods of FIG. 1 and FIGS. 2-4 may comprise an imaging sensor. The imaging sensor may be portable, stand-off (remote from or not worn by the subject), and/or worn by the subject (such as that described for the digital camera in U.S. Pat. No. 10,623,849, which is incorporated herein by reference in its entirety). A variety of imaging sensors are well known to those skilled in the art, including, but not limited to: CCD imagers, CMOS imagers, NMOS imagers, photodetector arrays, and the like. The ubiquitous nature of cameras in modern mobile electronics affords a variety of setups for imaging a subject who is also wearing a blood pressure monitor to generate a blood pressure measurement.

There are important benefits that may be afforded by utilizing an imaging sensor as the PPG sensor. Simultaneous data may be obtained from multiple distinct body locations (i.e., to map-out biometric estimations throughout the body). Also, simultaneous data at multiple wavelengths of electromagnetic energy (whether the photons are in the visible range or otherwise) may be obtained. For example, embodiments of the present invention described herein, when utilized with an imaging sensor as the PPG sensor, may be used to continuously estimate blood pressure for a subject at multiple body locations simultaneously, with measurement data provided by at least one blood pressure monitor. In one embodiment, the subject may be wearing a blood pressure cuff at the arm (e.g., via a brachial blood pressure cuff), and this measurement data is then fed to the system and methods of FIG. 1 and FIGS. 2-4. Processing the imaging data and the BP measurement data, the adaptive predictive model (e.g., 300, FIG. 8) may be configured to estimate continuous BP for multiple body locations simultaneously. In one exemplary implementation, the PPG data collected from the brachial artery region (where the BP measurement is collected) may be processed to generate an estimation of BP at that region of the body, and this relationship may be extrapolated to the other body regions in the view of the imaging sensor, such that blood pressure across various regions of the body may be estimated continuously. In turn, the biometric estimates from multiple body locations can be further processed to generate an overall hemodynamic assessment for the subject. For example, irregularities or nonuniformities of blood pressure may be indicative of a poor blood flow (i.e., a blood vessel blockage, hemorrhaging, a vascular issue, or the like). Moreover, the relative blood pressure at the heart (central blood pressure) vs. the blood pressure at the extremities may be processed to indicate peripheral resistance to blood flow or other cardiovascular issues. Additionally, an assessment of the temporal dynamics between central and peripheral blood pressure can be used to assess pulse transit time (PTT) and pulse wave velocity (PWV) for the subject.

It should also be noted that the blood pressure measurement device itself may be a remote imaging sensor in this invention. And in such case the benefit of a wearable PPG sensor may be the ability to passively assess blood pressure (via the blood pressure estimation) following BP measurements taken via video (collected via the imaging sensor). Reversely, the blood pressure measurement device in this invention itself may be a wearable PPG sensor (i.e., that has been optimized for measuring or estimating BP) or a blood pressure cuff, and the biometric sensor may be an imaging sensor. In such case, a key benefit of this approach may be that a remote imaging system can continuously and passively (in the background without requiring a change in subject behavior) improve BP estimations for the subject as new data from the blood pressure measurements continues to improve the adaptive predictive model.

### Alternatives to PPG

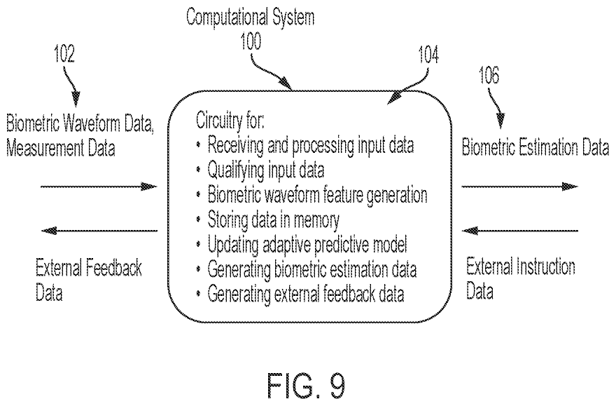

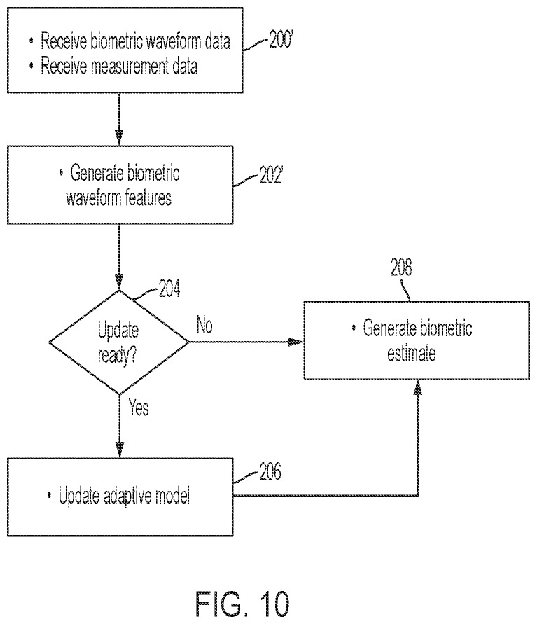

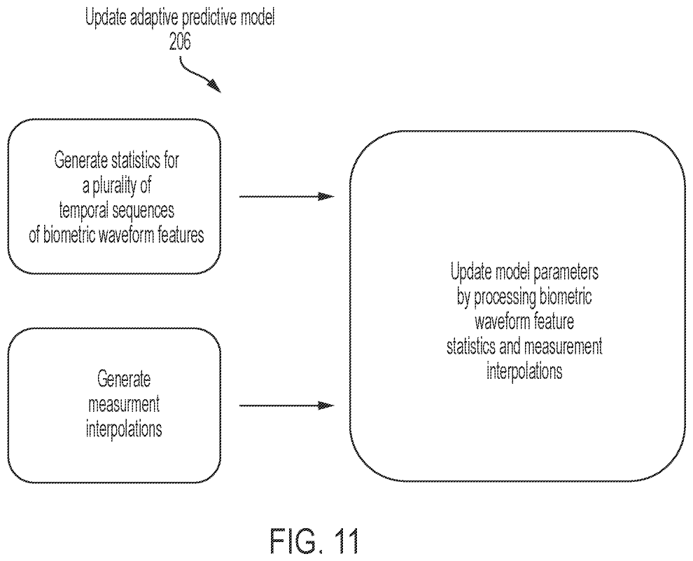

The invention may also be applicable for the case where the biometric sensor data is not PPG sensor data but rather a different sensor modality (or a combination of these modalities in a sensor fusion approach), such as sensor data that is electromagnetic, auscultatory, electrical, magnetic, mechanical, thermal, or the like. In such case, the more general invention is presented in FIG. 9, FIG. 10, and FIG. 11. For the invention to work suitably for these other sensor modalities, in context of accurately and continuously estimating subject BP, it is important that the sensor modalities enable a continuous stream of waveform data and that the rate of change of feature statistics is comparable to that associated with PPG waveform data. Because there are auscultatory, mechanical, and thermal variations from the body that are time-coincident with PPG waveforms, these particular modalities (in comparison with the list of modalities above) may be most suitable for estimating blood pressure via the invention herein.

### Definition of Predicting

In some cases, the estimations generated by the adaptive predictive model may be estimations of what the model predicts the estimations truly are in real-time. However, in some cases, the estimations generated by the adaptive predictive model may be estimations of what the model predicts the estimations will be in the impending future. One method of achieving this is via an adaptive predictive model that is tuned to generate future biometric estimations (for a future point in time) given a set of real-time data as opposed to current biometric estimations. Another method of achieving this may be to generate current biometric estimations, using methods as those outlined above, but then applying an additional layer to the model such that trends in past and current biometric estimations are further processed to generate predictions of future biometric estimations. Other approaches may be utilized.

A key benefit of predicting future biometric estimations (as opposed to current biometric estimations) may be that subjects wearing embodiments of the present invention may be informed that future undesirable biometric values may be imminent, such that they may take prophylactic measures to prevent these undesirable values. For example, a subject managing diabetes may benefit from knowing that their blood glucose levels are about to rise/drop sharply, enabling them to take a prophylactic dose of insulin/glucose to prevent this negative outcome. Similarly, a subject managing hypertension may benefit from knowing that their blood pressure is about to rise/drop sharply, enabling them to medicate accordingly.

Example embodiments are described herein with reference to block diagrams and flow diagrams. It is understood that a block of the block diagrams and flow diagrams, and combinations of blocks in the block diagrams and flow diagrams, can be implemented by computer program instructions that are performed by one or more computer circuits, such as electrical circuits having analog and/or digital elements. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and flow diagrams, and thereby create means (functionality) and/or structure for implementing the functions/acts specified in the block diagrams and flow diagrams.

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and flow diagrams.

A tangible, non-transitory computer-readable medium may include an electronic, magnetic, optical, electromagnetic, or semiconductor data storage system, apparatus, or device. More specific examples of the computer-readable medium would include the following: a portable computer diskette, a random access memory (RAM) circuit, a read-only memory (ROM) circuit, an erasable programmable read-only memory (EPROM or Flash memory) circuit, a portable compact disc read-only memory (CD-ROM), and a portable digital video disc read-only memory (DVD/BlueRay).

The computer program instructions may also be loaded onto a computer and/or other programmable data processing apparatus to cause a series of operational steps to be performed on the computer and/or other programmable apparatus to produce a computer-implemented process such that the instructions which execute on the computer or other programmable apparatus provide steps for implementing the functions/acts specified in the block diagrams and flow diagrams. Accordingly, embodiments of the present invention may be embodied in hardware and/or in software (including firmware, resident software, micro-code, etc.) that runs on a processor such as a digital signal processor, which may collectively be referred to as “logic”, “circuitry”, “a module”, “an engine” or variants thereof.

It should also be noted that the functionality of a given block of the block diagrams and flow diagrams may be separated into multiple blocks and/or the functionality of two or more blocks of the block diagrams and flow diagrams may be at least partially integrated. Finally, other blocks may be added/inserted between the blocks that are illustrated. Moreover, although some of the diagrams include arrows on communication paths to show a primary direction of communication, it is to be understood that communication may occur in the opposite direction to the depicted arrows.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

1-22. (canceled)

23. A method of improving blood glucose estimation accuracy of an adaptive predictive model, the method comprising the following steps performed by at least one processor:
a) receiving, within a receiving period, real-time PPG data from a PPG sensor attached to a subject and a blood glucose measurement from a blood glucose monitoring device;
b) generating features from the received PPG data;
c) storing the features and the blood glucose measurement; and
d) updating one or more parameters of the adaptive predictive model in real-time by processing the stored features in context with the stored blood glucose measurement, wherein the updated one or more parameters improves the blood glucose estimation accuracy of the adaptive predictive model.

24. The method of claim 23, further comprising repeating steps a)-d) over one or more subsequent time periods.

25. The method of claim 23, further comprising:
generating a blood glucose estimation for the subject via the adaptive predictive model;
determining whether the generated blood glucose estimation is above or below a threshold;
responsive to determining that the generated blood glucose estimation is above or below the threshold, receiving another measurement of blood glucose via the blood glucose monitoring device; and
updating the one or more parameters of the adaptive predictive model in real-time.

26. The method of claim 23, wherein generating features from the received PPG data comprises generating features at feature generation intervals within the receiving period via a sliding time window.

27. The method of claim 23, wherein updating the one or more parameters of the adaptive predictive model further comprises processing the stored blood glucose measurement and a previously stored blood glucose measurement, and generating an interpolation between the stored blood glucose measurement and the previously stored blood glucose measurement.

28. The method of claim 27, wherein processing the stored blood glucose measurement and the previously stored blood glucose measurement further comprises processing a plurality of previously stored blood glucose measurements.

29. The method of claim 28, wherein processing the stored blood glucose measurement and the previously stored blood glucose measurement further comprises generating an interpolation of expected blood glucose measurements.

30. The method of claim 23, wherein the PPG sensor comprises an imaging sensor.

31. The method of claim 23, wherein the adaptive predictive model comprises one of a regression model, a machine learning model, or a classifier model.

32. The method of claim 23, wherein the features and the blood glucose measurement are stored in a data buffer.

33. The method of claim 32, wherein the data buffer comprises a FIFO (first-in-first-out) buffer.

34. The method of claim 23, wherein processing the stored features in context with the stored blood glucose measurement comprises processing a function of at least one of the stored features.

35. The method of claim 23, wherein processing the stored features in context with the stored blood glucose measurement comprises calculating statistical information for a temporal sequence of at least one of the stored features.

36. The method of claim 23, wherein processing the stored features in context with the stored blood glucose measurement comprises calculating statistical information for a plurality of temporal sequences of at least one of the stored features.

37. The method of claim 23, wherein processing the stored features in context with the stored blood glucose measurement comprises calculating weighted statistical information for a plurality of temporal sequences of at least one of the stored features.

38. A system for improving blood glucose estimation accuracy of an adaptive predictive model, the system comprising at least one processor configured to:
receive, within a receiving period, real-time PPG data from a PPG sensor attached to a subject and a blood glucose measurement from a blood glucose monitoring device;
generate features from the received PPG data;
store the features and the blood glucose measurement; and
update one or more parameters of the adaptive predictive model in real-time by processing the stored features in context with the stored blood glucose measurement, wherein the updated at least one parameter improves blood glucose estimation accuracy of the adaptive predictive model.

39. The system of claim 38, wherein the at least one processor is further configured to:
generate a blood glucose estimation via the adaptive predictive model;
determine whether the generated blood glucose estimation is above or below a threshold; and
in response to determining that the generated blood glucose estimation is above or below the threshold, update the one or more parameters of the adaptive predictive model in real-time.

40. The system of claim 39, wherein the at least one processor is further configured to send an alert to a remote device that the generated blood glucose estimation is above or below the threshold.

41. The system of claim 38, wherein the adaptive predictive model comprises one of a regression model, a machine learning model, or a classifier model.
